(19) United States
(12) Patent Application Publication
(10) Pub. No.: US 2024/0157063 A1
(43) Pub. Date: May 16, 2024

(54) DRUG DELIVERY DEVICE

(71) Applicant: HCMed Innovations Co., LTD., Taipei City (TW)

(72) Inventors: Chieh-Sheng Cheng, Taipei City (TW); JUI-SHUI CHEN, New Taipei City (TW); YI-HUNG WANG, New Taipei City (TW)

(21) Appl. No.: 18/389,243

(22) Filed: Nov. 14, 2023

Related U.S. Application Data
(60) Provisional application No. 63/424,950, filed on Nov. 14, 2022.

(30) Foreign Application Priority Data
Nov. 2, 2023 (CN) 202311451605.8

(51) Int. Cl. A61M 11/00

(52) U.S. Cl. CPC A61M 11/007; A61M 2205/02; A61M 2205/106; A61M 2205/3327; A61M 2205/50

(57) ABSTRACT

A drug delivery device including a main housing and a drug delivery module is provided. The main housing has an internal space. The drug delivery module is disposed in the internal space so as to be isolated from an external environment. The drug delivery module includes a drug bottle that contains a liquid drug and a driver that is connected to the drug bottle. The driver is configured to push the liquid drug to pass through a drug nebulization structure of the drug bottle such that the liquid drug is nebulized into a nebulized drug.

### CROSS-REFERENCE TO RELATED PATENT APPLICATION

This application claims the benefit of priority to China Patent Application No. 202311451605.8, filed on Nov. 2, 2023, in the People's Republic of China. The entire content of the above identified application is incorporated herein by reference.

This application claims the benefit of priority to the U.S. Provisional Patent Application Ser. No. 63/424,950, filed on Nov. 14, 2022, which application is incorporated herein by reference in its entirety.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a drug delivery device, and more particularly to a drug delivery device having a driver that is disposed in a separate space.

### BACKGROUND OF THE DISCLOSURE

Nebulized inhalation therapy uses a nebulization device to disperse a drug into particles suspended in the gas, so that the drug can enter the respiratory tract through breathing to improve the efficiency of drug delivery and achieve therapeutic purposes.

An existing handheld nebulizer device is a manual spraying nebulizer with a spring or a screw rod as the compression structure. In order to use the device, it is necessary to apply pressure to a drug inside a drug bottle in manners such as manual rotation such that part of the drug enters a temporary storage chamber. Then, the drug stored in the temporary storage chamber is sprayed out for a user to inhale by pressing a button.

However, the existing handheld nebulizer device is limited by the pressure that can be applied to the drug and the holding space of the temporary storage chamber, such that the amount of drug that is dispensed each time is less than 0.03 cc. Therefore, the total amount of the drug for a single treatment (e.g., 1 cc) requires several manual operations (i.e., about thirty manual rotations and press-releases) to complete. In addition, the compression structures such as springs or screw rods need to be linked with components exposed in the external environment for allowing the user to operate them. The compression structures have a more complicated structure and risk operational failure due to the influences from the external environment. Furthermore, drug delivery through manual pressure tends to be inaccurate and unstable.

Therefore, how to improve on the defects of the nebulized drug delivery device that is unable to accurately and stably deliver the drug by improving the structural design has become one of the important issues to be addressed in this industry.

### SUMMARY OF THE DISCLOSURE

In response to the above-referenced technical inadequacies, the present disclosure provides a drug delivery device.

In order to solve the above-mentioned problems, one of the technical aspects adopted by the present disclosure is to provide a drug delivery device. The drug delivery device includes a main housing and a drug delivery module. The main housing has an internal space. The drug delivery module is disposed in the internal space so as to be isolated from an external environment. The drug delivery module includes a drug bottle that contains a liquid drug and a driver that is connected to the drug bottle. The driver is configured to push the liquid drug to pass through a drug nebulization structure of the drug bottle such that the liquid drug is nebulized into a nebulized drug.

In one of the possible or preferred embodiments, the driver is connected to the drug bottle via a piston, and the piston is moved along an inner wall of the drug bottle by a thrust provided by the driver.

In one of the possible or preferred embodiments, the drug bottle and the piston define a first space, and the piston and the driver define a second space. The second space is an independent enclosed space.

In one of the possible or preferred embodiments, the drug bottle is detachably assembled to the driver, the drug bottle has a first piston, the driver has a second piston, and the second piston pushes the first piston through a thrust provided by the driver such that the first piston is moved along an inner wall of the drug bottle.

In one of the possible or preferred embodiments, a third space is defined between the first piston and the second piston, and the third space is an independent enclosed space.

In one of the possible or preferred embodiments, the driver is a pneumatic pump, a micro-motor, or an electromagnetic reciprocating structure.

In one of the possible or preferred embodiments, the drug nebulization structure is a micro-nozzle or a filtering screen.

In one of the possible or preferred embodiments, the drug delivery device further includes a control module. The control module includes a circuit board, a position sensor, and a battery. The circuit board drives the driver to execute a driving program, the position sensor senses a position of a piston driven by the driver, and the battery stores electrical power for driving the piston.

In one of the possible or preferred embodiments, the drug delivery device further includes a support base. The support base encloses the drug bottle and supports the control module.

In one of the possible or preferred embodiments, the drug delivery device further includes a waterproof module. The waterproof module is at least disposed on a side of the support base adjacent to the nozzle, and the waterproof module surrounds a periphery of the side.

Therefore, in the drug delivery device provided by the present disclosure, by virtue of “a main housing having an internal space, and a drug delivery module being disposed in the internal space so as to be isolated from an external environment,” and “the drug delivery module including a drug bottle that contains a liquid drug and a driver that is connected to the drug bottle, and the driver being configured to push the liquid drug to pass through a drug nebulization structure of the drug bottle such that the liquid drug is nebulized into a nebulized drug,” the delivery stability of the drug delivery device can be improved. Furthermore, because the driver is able to continuously push the piston according to settings, the drug delivery device of the present disclosure is also able to increase a dosage of a single delivery, thereby simplifying an operation process for users.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a schematic perspective view of a drug delivery device according to a first embodiment of the present disclosure;

FIG. 2 is a schematic exploded view of the drug delivery device according to the first embodiment of the present disclosure;

FIG. 3 is a cross-sectional view taken along line III-III of FIG. 1;

FIG. 4 is a schematic perspective view of a drug delivery device according to a second embodiment of the present disclosure;

FIG. 5 is a schematic exploded view of the drug delivery device according to the second embodiment of the present disclosure;

FIG. 6 is a cross-sectional view taken along line VI-VI of FIG. 4;

FIG. 7 is a schematic perspective view of a drug delivery device according to a third embodiment of the present disclosure;

FIG. 8 is a schematic exploded view of the drug delivery device according to the third embodiment of the present disclosure; and

FIG. 9 is a cross-sectional view taken along line IX-IX of FIG. 7.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a,” “an” and “the” includes plural reference, and the meaning of “in” includes “in” and “on.” Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first,” “second” or “third” can be used to describe various components, signals or the like, which are for distinguishing one component/signal from another one only, and are not intended to, nor should be construed to impose any substantive limitations on the components, signals or the like.

### First Embodiment

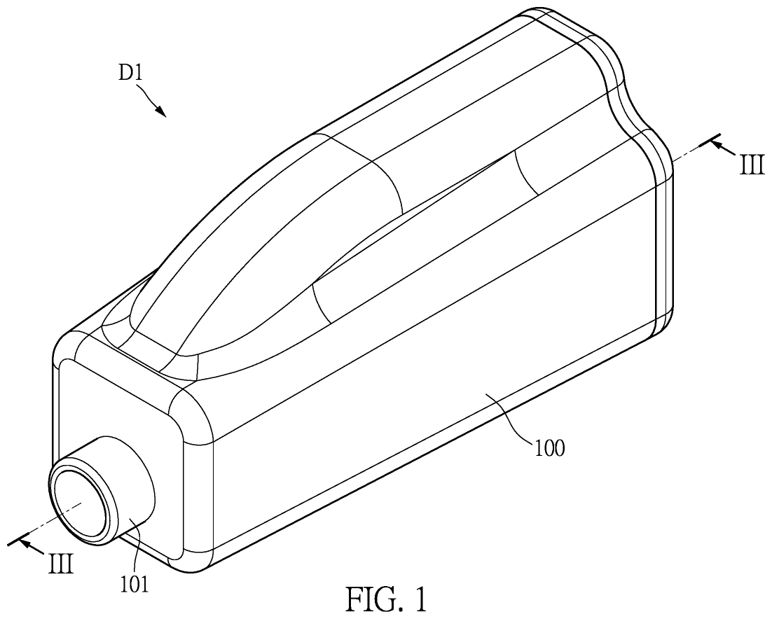

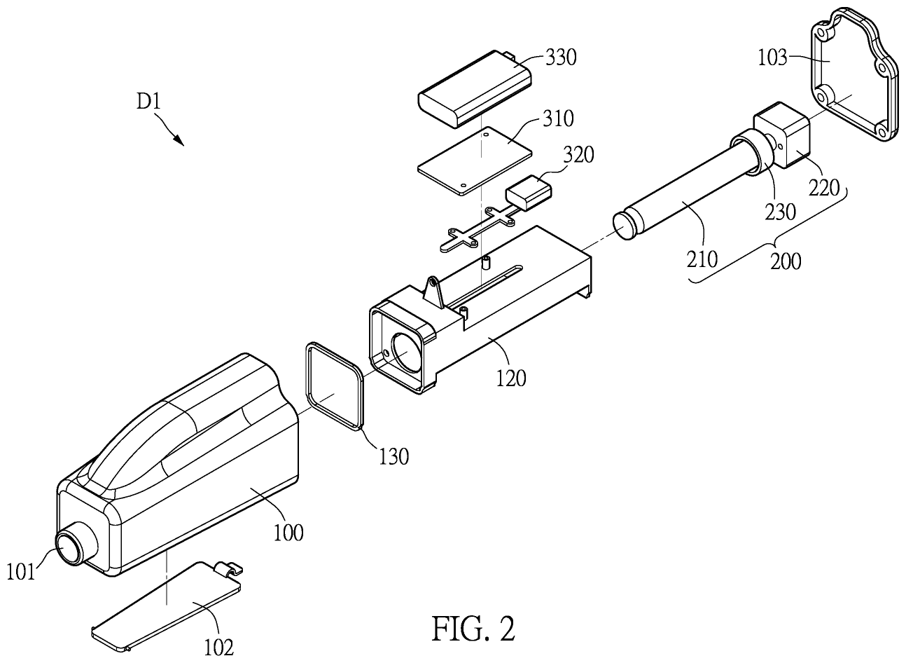

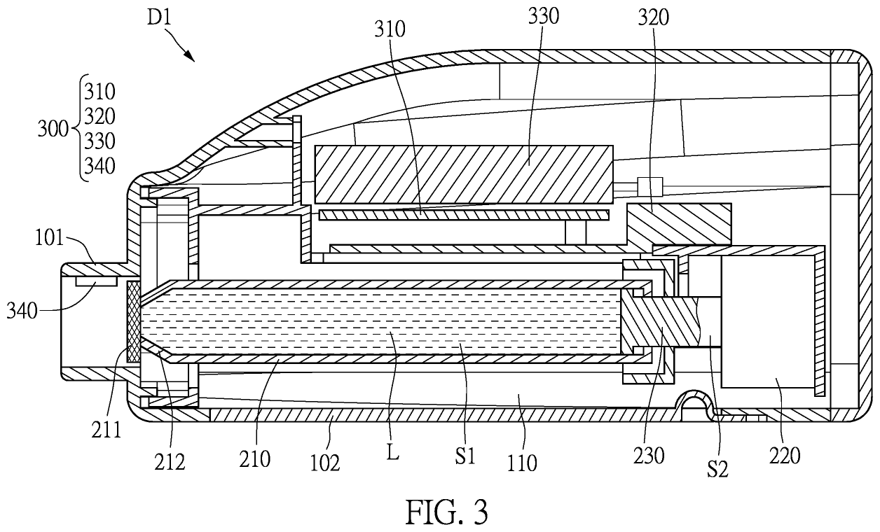

Referring to FIG. 1 to FIG. 3, a first embodiment of the present disclosure provides a drug delivery device D1. The drug delivery device D1 includes a main housing 100 having an internal space 110 and a drug delivery module 200 that is installed in the internal space 110. The drug delivery module 200 is installed in the internal space 110 so as to be isolated from an external environment. The main housing 100 has a nozzle 101. The nozzle 101 can be integrally formed together with the main housing 100 or can be detachably installed on the main housing 100. When the nozzle 101 is able to be detachably installed on the main housing 100, a user can install a corresponding nozzle 101 on the main housing 100 according to different requirements.

The drug delivery module 200 includes a drug bottle 210 that contains a liquid drug L and a driver 220 that is connected to the drug bottle 210. Furthermore, the drug bottle 210 has a drug nebulization structure 211. The drug nebulization structure 211 is disposed on a side of the drug bottle 210 adjacent to the nozzle 101. For example, the drug nebulization structure 211 can be a micro-nozzle or a filtering screen. However, the aforementioned example is only one of the practical embodiments, and is not intended to limit the scope of the present disclosure. In the present disclosure, when the driver 220 provides a pushing force to push the liquid drug L to pass through the drug nebulization structure 211, the liquid drug L can be nebulized into a nebulized drug (also referred to as an aerosol). Therefore, the present disclosure does not require an additional vibration module to drive the filtering screen for nebulizing the liquid drug L.

Specifically, the driver 220 includes a piston 230, and a front end of the piston 230 can be disposed in the drug bottle 210 and be moved along an inner wall of the drug bottle 210. In other words, the drug bottle 210 can be connected with the driver 220 via the piston 230. The piston 230 can be moved toward the nozzle 101 along an interior of the drug bottle 210 through the pushing force provided by the driver 220. The piston 230 then pushes the liquid drug L to pass through the drug nebulization structure 211 such that the liquid drug L is nebulized into the nebulized drug.

In this embodiment, the drug bottle 210 and the driver 220 are assembled into a single module, and the driver 220 is a disposable drive, such that the driver 220 and the drug bottle 210 can be detached and replaced together. The drug bottle 210 and the piston 230 can be used to define a first space S1 (i.e., a space for accommodating the liquid drug L), and a space between the piston 230 and the driver 220 can be used to define a second space S2. Because the driver 220 of the present disclosure is not a spring or a screw rod that is communicated with an external environment for being manually operated by the user, the second space S2 between the piston 230 and the driver 220 does not communicate with the external environment. In other words, regardless of how the driver 220 drives the piston 230, the second space S2 between the piston 230 and the driver 220 remains to be an independent enclosed space, and is not in spatial communication with the first space S1 or the external environment.

The drug delivery device D1 of the present disclosure can further include a control module 300. The control module 300 can be disposed in a portion of an internal space of the main housing 100 for driving the driver 220, and the portion of the internal space is different from a space occupied by the drug delivery module. The control module 300 can include a circuit board 310 and a battery 330. The circuit board 310 drives the driver 220 to execute various driving programs to control the movement of the piston 230, and the battery 330 is used to store an electric power for driving the piston 230 to perform the therapy for multiple times.

In the present disclosure, the driver 220 can be a pneumatic pump. In other words, an air is used as a source of pressure to push the piston 230. Specifically, the driver 220 can contain an electrically conductive liquid, and when the control module 300 conducts electricity to the electrically conductive liquid, a portion of the electrically conductive liquid is gasified from a liquid state to a gas state. Therefore, a pressure is produced in the second space S2 that is enclosed, and the gas generated from gasification can be used as a pressure source for pushing the piston 230.

When the piston 230 is pushed by the pressure of the gas and is moved toward the nozzle 101, the piston 230 inside of the drug bottle 210 is moved along the inner wall of the drug bottle 210 to push the liquid drug L toward the nozzle 101. Because an amount of the gas in the second space S2 is dependent on an intensity of an electric current, the control module 300 can control the intensity of the electric current such that a pressure from the pressure source of the driver 220 is adjusted. Therefore, a displacement of the piston 230 is controlled to achieve an effect of accurate drug delivery.

In the present disclosure, the driver 220 can also be a micro-motor that is able to accurately control a movement distance of the piston 230, so as to achieve a similar effect of accurate drug delivery.

### Second Embodiment

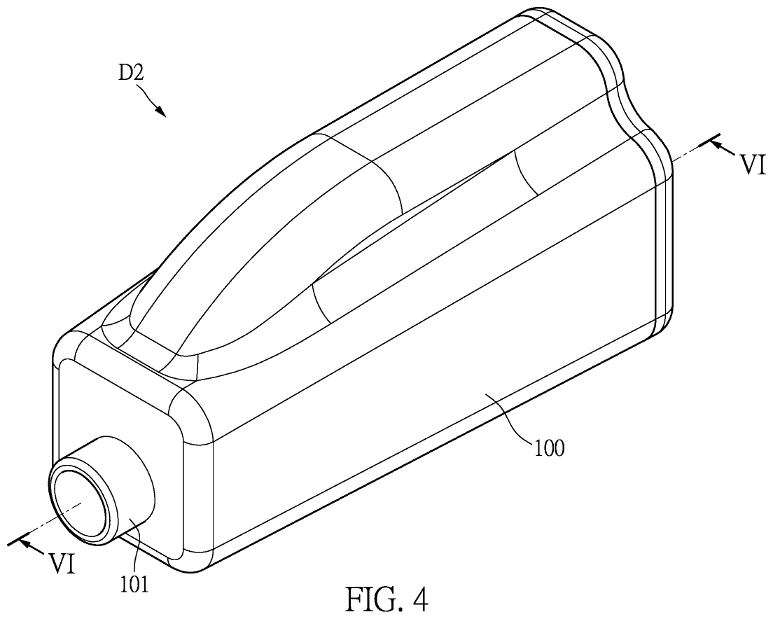

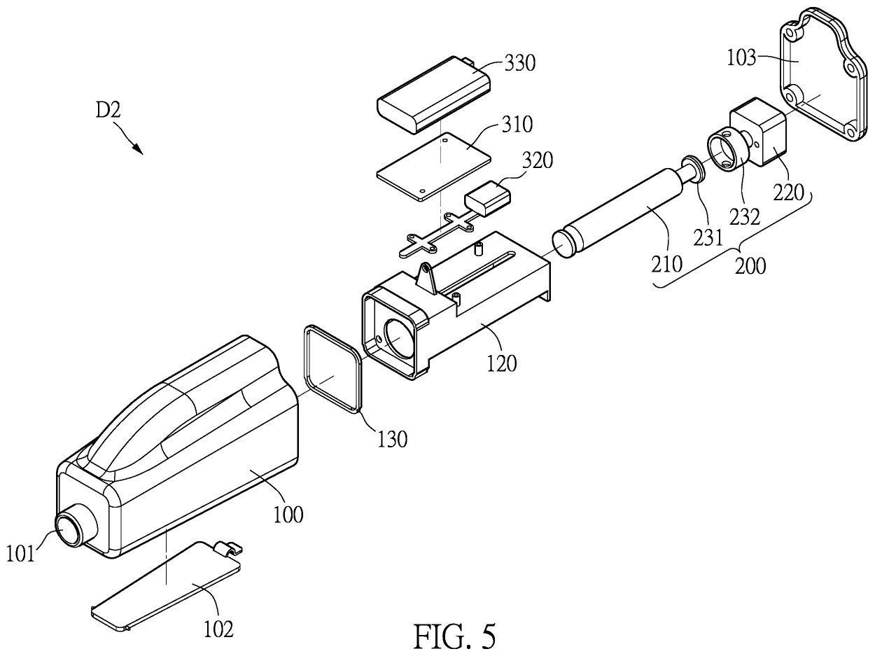

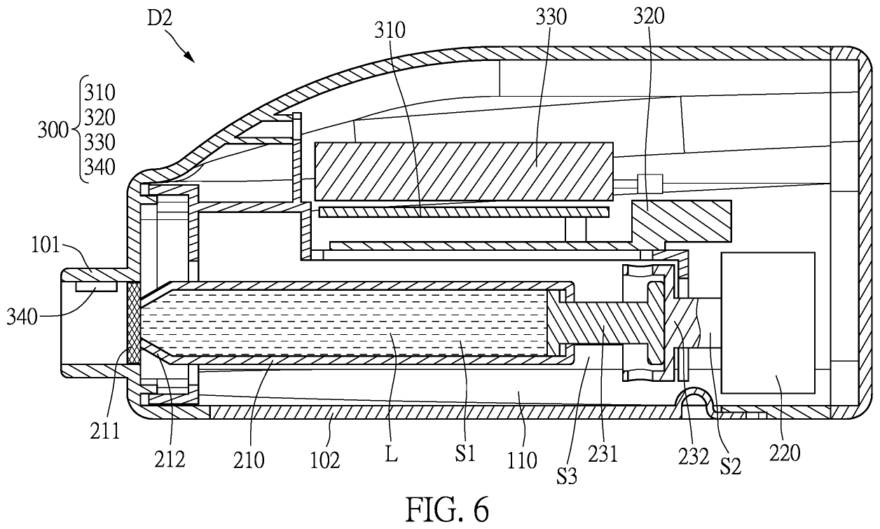

Referring to FIG. 4 to FIG. 6, a second embodiment of the present disclosure provides a drug delivery device D2. A main difference between the second embodiment and the first embodiment is that, the drug bottle 210 of this embodiment is detachably assembled to the driver 220. In other words, the drug bottle 210 can be separately detached and replaced, and the driver 220 can be configured as a fixed pressure source.

In detail, the drug bottle 210 of the second embodiment has a first piston 231 that is configured to push the liquid drug L in the drug bottle 210. The driver 220 has a second piston 232 that is configured to push the first piston 231. In other words, the drug bottle 210 is detachably assembled to the driver 220 by the first piston 231 abutting against the second piston 232.

After the drug bottle 210 is assembled to the driver 220, a third space S3 can be defined between the first piston 231 and the second piston 232. Because the third space S3 is located within the internal space 110, the third space is also an independent enclosed space that does not communicate with the external environment. Thus, the driver 220 provides a pushing force to the second piston 232, such that the second piston 232 can be moved in the third space S3 toward the first piston 231 for pushing the first piston 231. Therefore, the first piston 231 can push the liquid drug L in the drug bottle 210 along the inner wall of the drug bottle 210, such that the liquid drug L is pushed to pass through the drug nebulization structure 211 to produce the nebulized drug.

In this embodiment, the driver 220 can be a pneumatic pump or a micro-motor. The driver 220 is used to control a micro-movement of the second piston 232, so as to achieve the effect of accurate drug delivery.

In one embodiment, the control module 300 further includes a position sensor 320 that can be disposed on a movement path of the first piston 231 and the second piston 232 so as to sense a movement position of the first piston 231 and the second piston 232. For example, the position sensor 320 can be an optical sensor. Therefore, by the position sensor 320 sensing a position of the first piston 231 and/or the second piston 232 by optical sensing, and the control module 300 adjusting a movement of the second piston 232 by controlling the driver 220, a dosage of the nebulized drug generated after the liquid drug L is pushed by the first piston 231 can be accurately calculated.

In one embodiment of the present disclosure, the circuit board 310, the position sensor 320, and the battery 330 can be arranged to be parallel to each other for reducing an overall volume of the control module 300, but the present disclosure is not limited thereto.

Furthermore, the control module can further include a pressure sensor 340. The pressure sensor 340 can be disposed on the nozzle 101 for sensing a change in pressure when the user breathes. For example, when the user inhales through the nozzle 101, the pressure sensor 340 can sense a negative pressure and transmit an activation signal through the circuit board 310 to correspondingly activate the driver 220. Therefore, the first piston 231 is pushed by the second piston 232 to further push and nebulize the liquid drug L. In other words, the drug delivery device D2 of the present disclosure can activate the driver 220 via breath sensing. At this time, parameters obtained by the pressure sensor 340 are used to determine whether to continue or to stop pushing the second piston 232, so that the activation of the driver 220 is synchronized with the inhalation of the user. Therefore, a nebulization phase of the liquid medication L falls within the inhalation of the user to prevent wasting of the liquid medication L.

A flow guiding structure 212 can be formed on a front end of the drug bottle 210 to guide a flow of the liquid drug L. For example, the flow guiding structure 212 can be a structure having a width that is gradually narrowed along a direction toward the nozzle 101. When the first piston 231 continuously pushes the liquid drug L in the drug bottle 210, the flow guiding structure 212 enables the liquid drug L to move in a congregated manner toward the drug nebulization structure 211. At this time, a part of the liquid drug L located in the front end of the drug bottle 210 is pressed by an increased pressure, such that a nebulization efficiency of the liquid drug L passing through the drug nebulization structure 211 is further improved. However, the aforementioned example is only one of the implementations of the present disclosure, and the present disclosure is not limited thereto.

### Third Embodiment

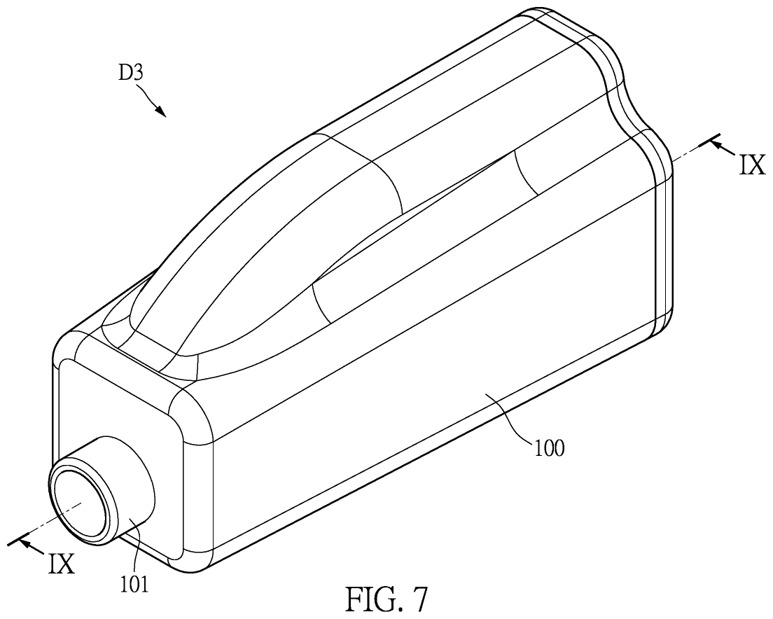

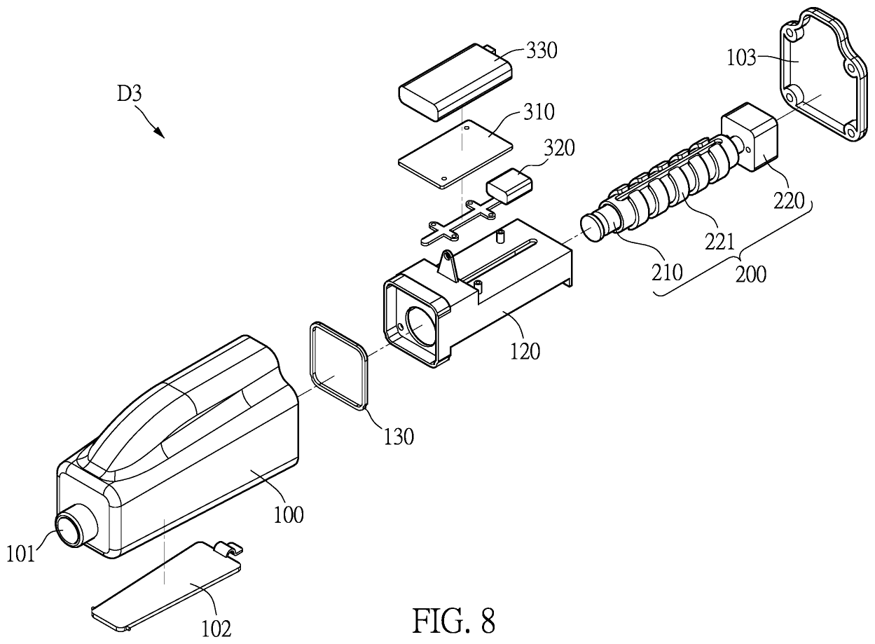

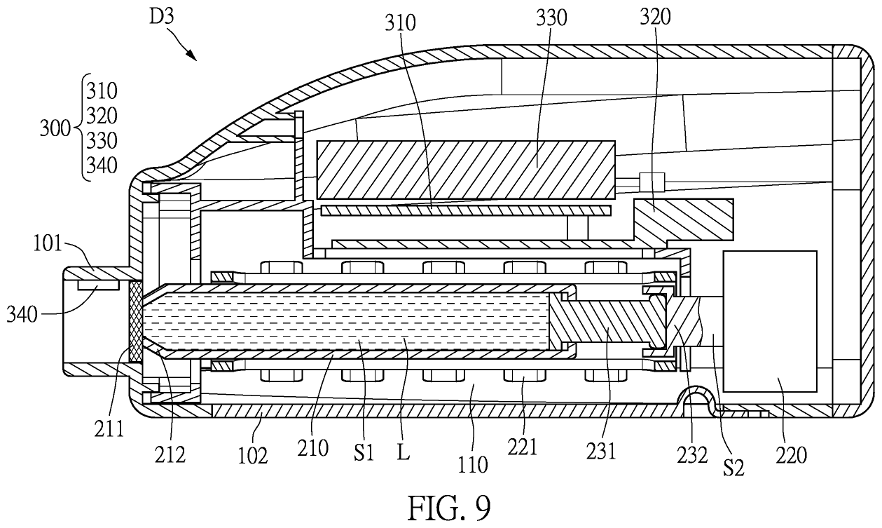

Referring to FIG. 7 to FIG. 9, a third embodiment of the present disclosure provides a drug delivery device D3. A main difference between the third embodiment and the second embodiment is that, the driver 220 of this embodiment is an electromagnetic reciprocating structure. Specifically, the driver 220 that is the electromagnetic reciprocating structure is implemented by a plurality of induction coils 221 surrounding a periphery of the first space S1 defined by the drug bottle 210 and the piston 230 and/or a periphery of the second space S2 defined by the piston 230 and the driver 220. Furthermore, the drug bottle 210 includes a first piston 231, the driver 220 includes a second piston 232, and the second piston 232 has opposite magnetic poles. When an electric current passes through the induction coils 221 to generate a magnetic field, the second piston 232 having the magnetic poles can be moved, and the second piston 232 synchronously drive the first piston 231 to push the liquid drug L of the drug bottle 210 to pass through the drug nebulization structure 211. Because only a small amount of the electric current is required to pass through the induction coils 221 to generate a magnetic field, the configuration of the electromagnetic reciprocating structure enables the movement distance of the second piston 232 to be accurately controlled in a non-contact manner.

The drug delivery devices D1 to D3 of the aforementioned embodiments can further include a support base 120 and a waterproof module 130 that are disposed in the internal space 110. In this embodiment, the support base 120 can be a hollow strip structure that surrounds the drug bottle 210 and supports the control module 300. In other words, the drug bottle 210 and the control module 300 can be spaced apart by the support base 120 so as to prevent the liquid drug L in the drug bottle 210 that is spilled out from leaking to the control module 300. The waterproof module 130 can be disposed on a side of the support base 120 adjacent to the nozzle 101. When the user performs a therapy treatment, the waterproof module 130 can prevent the liquid drug L that is nebulized from leaking to the control module 300. In other embodiments, the support base 120 can surround both the drug bottle 210 and the driver 220.

In other embodiments, the waterproof module 130 can further surround the support base 120 to provide an improved waterproofing effect. However, as long as the liquid drug L can be prevented from leaking to the control module 300, an arrangement of the waterproof module 130 is not limited in this embodiment.

In one embodiment of the present disclosure, the waterproof module 130 can further include a bottom cap 102 and a rear cap 103. The bottom cap 102 can be engaged to the main housing 100 to correspond to the internal space 110 in position, so as to enable the detachment of the bottom cap 102 and replacement of the drug bottle 210 in the internal space 110. The rear cap 103 can be disposed on one side of the main housing 100 opposite to the nozzle 101. When the drug delivery devices D1 to D3 are assembled, the rear cap 103 can be threadedly fixed onto the main housing 100 by using a screw, such that the rear cap 103 and the main housing 100 jointly form an internal space that accommodates the drug bottle 210, the driver 220, and the control module 300.

### Beneficial Effects of the Embodiments

In conclusion, in the drug delivery device provided by the present disclosure, by virtue of “a main housing having an internal space, and a drug delivery module being disposed in the internal space so as to be isolated from an external environment,” and “the drug delivery module including a drug bottle that contains a liquid drug and a driver that is connected to the drug bottle, and the driver being configured to push the liquid drug to pass through a drug nebulization structure of the drug bottle such that the liquid drug is nebulized into a nebulized drug,” the delivery stability of the drug delivery device can be improved.

Furthermore, the drug delivery device of the present disclosure uses the control module to control the driver located in a separate space, and the movement distance of the piston can be controlled to estimate a dosage of drug to be delivered, thereby avoiding limitations of human operation and environmental errors, and avoiding the need to form a temporary storage chamber. Therefore, the effect of accurate one-time drug delivery (e.g., 0.03 cc or more) can be achieved. Specifically, the drug delivery device of the present disclosure is capable of accurately delivering a single dose of a drug in an amount ranging from 0.03 cc to 1 cc.

In addition, by the configuration of a micro-nozzle or a filtering screen, the present disclosure enables the drug delivery device to directly nebulize the drug when the piston pushes the liquid drug, so that the drug delivery device of the present disclosure does not need an additional vibration module to nebulize the liquid drug. Therefore, the volume of the drug delivery device can be effectively reduced, and manufacturing costs can be saved.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A drug delivery device, comprising:
a main housing having an internal space; and
a drug delivery module disposed in the internal space so as to be isolated from an external environment;
wherein the drug delivery module includes a drug bottle that contains a liquid drug and a driver that is connected to the drug bottle, and wherein the driver is configured to push the liquid drug to pass through a drug nebulization structure of the drug bottle such that the liquid drug is nebulized into a nebulized drug.

2. The drug delivery device according to claim 1, wherein the driver is connected to the drug bottle via a piston, and the piston is moved along an inner wall of the drug bottle by a thrust provided by the driver.

3. The drug delivery device according to claim 2, wherein the drug bottle and the piston define a first space, and the piston and the driver define a second space, and wherein the second space is an independent enclosed space.

4. The drug delivery device according to claim 1, wherein the drug bottle is detachably assembled to the driver, the drug bottle has a first piston, the driver has a second piston, and the second piston pushes the first piston through a thrust provided by the driver, such that the first piston is moved along an inner wall of the drug bottle.

5. The drug delivery device according to claim 4, wherein a third space is defined between the first piston and the second piston, and the third space is an independent enclosed space.

6. The drug delivery device according to claim 1, wherein the driver is a pneumatic pump, a micro-motor, or an electromagnetic reciprocating structure.

7. The drug delivery device according to claim 1, wherein the drug nebulization structure is a micro-nozzle or a filtering screen.

8. The drug delivery device according to claim 1, further comprising a control module including a circuit board, a position sensor, and a battery, wherein the circuit board drives the driver to execute a driving program, the position sensor senses a position of a piston driven by the driver, and the battery stores electrical power for driving the piston.

9. The drug delivery device according to claim 8, further comprising a support base, wherein the support base encloses the drug bottle and supports the control module.

10. The drug delivery device according to claim 9, further comprising a waterproof module, wherein the waterproof module is at least disposed on a side of the support base adjacent to the nozzle, and the waterproof module surrounds a periphery of the side.
